(19) United States
(12) Patent Application Publication
(10) Pub. No.: US 2009/0105191 A1
(43) Pub. Date: Apr. 23, 2009

(54) Pharmaceutical and Dermatocosmetic Compositions Comprising Extract of Durio Zibenthinus

(76) Inventor: Howard Murad, Marina Del Rey, CA (US)

Correspondence Address: LOUIS C. PAUL, 420 East 61st Street, 8E, NEW YORK, NY, 10021, US

(21) Appl. No.: 12/085,907

(22) PCT Filed: Dec. 4, 2006
(86) PCT No.: PCT/US2006/046138

§ 371 Date: Jun. 2, 2008

Related U.S. Application Data
(60) Provisional application No. 60/741,466, filed on Dec. 2, 2005.

(51) Int. Cl. A61K 31/715; A61P 17/00

(52) U.S. Cl. 514/54

(57) ABSTRACT

The present invention relates to dermatocosmetic and pharmaceutical compositions comprising compounds obtained from Durio zibethinus and their use in increasing phase angle and reducing the signs and appearance of photoaging and biological aging.

### FIELD OF THE INVENTION

The present invention relates to dermatocosmetic and pharmaceutical compositions useful in reducing the signs and appearance of photoaging and biological aging. More particularly, the invention relates to the administration of therapeutically-effective levels of cosmetic and pharmaceutical compositions containing compounds obtained from the Durian plant (Durio zibethinus).

### BACKGROUND OF THE INVENTION

D. zibethinus is plant species of the family Bombacaceae native to Southeast Asia. It is reported to be rich in essential fatty acids (EFAs). According to Wong et al., approximately 60% of the total fatty acids in the pulp of the fruit (also known as the arrilus), is comprised of omega-3, omega-6 and omega-9 fatty acids. Anti-inflammatory, omega-3 fatty acid typically comprises about 50% of the total fatty acid content and is present in a ratio of greater than about 10:1 to pro-inflammatory omega-6 fatty acid. See, “Volatile Constituents of Durian,” Flav. Fragr. J., 10: 79-83 (1995).

The fruit of D. zibethinus also contains a particular distribution of sugars and sugar-derivatives (arabinose, rhamnose, fructose, glucose and galacturonic acid). Pongsamart, S. et al., “Novel water soluble antibacterial dressing of durian polysaccharide gel” Acta Horticulturae 678: 65-73 (2005). The comparative antioxidant activities of three fruits indigenous to Singapore—D. zibethinus, Nephelium lappaceum (rambutan), and Garcinia mangostana (mangosteen)—were reported in 2003 by Guan and Whiteman at the National University of Singapore 15^{th} Science Research Congress.

US Patent Application Publication Number 2005/0089499 to Cognis claims cosmetic/pharmaceutical active ingredients formed by adding a microorganism to a fermentation broth comprising plant constituents, which are broadly taught to include nodules, roots, leaves and preferably seeds and/or fruit seeds in size-reduced and/or pressed and/or extracted form. Durian is listed among 85 plant constituents disclosed in this publication; it is not, however, taught to be preferred. Durian extract is not listed in the 10^{th} Edition of the International Cosmetics Ingredient Dictionary and Handbook (Cosmetics and Toiletries Fragrance Association 2004).

Aging, both due natural biological processes and environmental stressors (e.g., ultraviolet radiation, pollutants), has been associated with impairment of cell membranes and connective tissue. With respect to the skin, barrier and regulatory functions are reduced. See Murad, The Cellulite Solution (St. Martin's Press, 2004). Biologically-aged skin is thinner, less elastic, and less resilient; it presents clinically as fine lines and deepening of facial expression lines. Photodamaged skin has a rough, leathery, yellowed appearance; clinically, photodamaged skin has coarse wrinkles and furrows. Whether as a consequence of environmental or intrinsic factors, with time, the skin loses water content and exhibits a decreased ability to heal and/or undergo repair. Additionally, with time, turnover of epidermal cells decreases with age.

Skin health (or impairment) can be measured directly by a variety of non-invasive techniques known to those of skill in the art. Commonly used metrics include skin moisture content, trans-epidermal water low, elasticity, blood flow, skin thickness, skin firmness and pH. Cell membrane integrity can be measured indirectly via bioelectrical impedance. Cells with intact membranes act as better capacitors, have higher capacitive reactance and, thus, higher measured phase angle values. Conversely, cells with a lower degree of cytoplasmic membrane integrity have a lower capacitance and demonstrate lower or decreased phase angle value. The latter measurement has been used both to diagnose disease and to demonstrate improvements in cytoplasmic membrane integrity as a result of therapeutic intervention.

There remains a long-felt but as unmet yet need for treatment modalities, particularly topical therapeutics, that help reduce the signs of aging while concomitantly helping to restore and/or support cell membrane integrity and proper connective tissue functioning. This need is met by the compositions and methods of treatment of the present invention.

### SUMMARY OF THE INVENTION

The present invention relates to dermatocosmetic and pharmaceutical compositions comprising therapeutically-effective amounts of a biologically-active extract of D. zibethinus, or fractions thereof, comprising (i) 9-octadecanoic acid in a ratio of at least about 2:3 to the total fatty acid content of the extract and (ii) a mixture of polysaccharides comprising arabinose, rhamnose, fructose, glucose and galacturonic acid. Compositions of the present invention are useful in the maintenance and enhancement of cell membrane integrity and connective tissue functioning. More particularly, compositions of the present invention are useful in preventing or treating the loss of body water content as well as reducing the appearance of fine lines and wrinkles and preventing or treating loss of skin firmness or skin elasticity, all of which are associated with biological aging and photoaging.

### DETAILED DESCRIPTION OF THE INVENTION

The present invention relates to dermatocosmetic and pharmaceutical compositions comprising therapeutically-effective amounts of a biologically-active extract of D. zibethinus, or fractions thereof, comprising (i) 9-octadecanoic acid in a ratio of at least about 2:3 to the total fatty acid content of the extract and (ii) a mixture of polysaccharides comprising arabinose, rhamnose, fructose, glucose and galacturonic acid. Preferably, 9-octadecanoic acid is present in a ratio of at least about 1:1 to the total fatty acid content of the extract.

In a preferred embodiment, the composition of the present invention further comprises a dermatologically-acceptable carrier.

In another preferred embodiment, the ratio of unsaturated to saturated fatty acids in the extract is greater than about 3:2.

The biologically-active extract of D. zibethinus, and/or fractions thereof, useful in this invention can be prepared by standard extraction techniques known to those of skill in the art. Suitable extraction vehicles include, but are not limited to, ethanol, methanol, ethyl acetate, acetone, chloroform and water, or any other solvent and water. The biologically-active fractions can be obtained from any portion of the plant, but preferably the extract is taken from the arillus. One method for producing a suitable extract for use in the present invention is described in U.S. Patent Application Publication No. 2003/0166608, the disclosure of which is incorporated herein by reference.

Compositions of the present invention may be administered in a number of dosage forms, preferably oral and topical. Topical dosage forms include solutions, colloidal dispersions, emulsions (oil-in-water or water-in-oil), suspensions, powders, creams, lotions, ointments, gels, foams, mousses, sprays and the like, made according to well-known methodologies in the art, including those found, for example, in Remington, The Science and Practice of Pharmacy (20^{th} Edition, 2000).

As will be appreciated by persons of ordinary skill in the art, dose and dose frequency of compositions of the present invention will vary among patients to account for, among other factors, age, weight, severity of condition(s) being treated. Typically, topical embodiments of the present invention can be applied directly to the skin in need of treatment in an amount of from about 0.1 mg/cm² to 2 mg/cm² of skin.

The term “therapeutically-effective amount” as used herein depends on the purity of the source material. For example, a crude extract is employed at a higher unit per weight concentration than a more pure extract.

The term “dermatologically-acceptable carrier” as used herein means a carrier that is suitable for topical application to the keratinous tissue, has good aesthetic properties, is compatible with the pharmaceutically active ingredients, and other optional components of the present invention, and does not produce irritation or other adverse health effects or safety concerns. The carrier can be in a wide variety of forms, including, but not limited to, oil-in-water emulsions, water-in-oil emulsions, water-in-silicone emulsions, silicone-in-water emulsions, water-in-oil-in-water, and oil-in-water-in-oil emulsions, and oil-in-water-in-silicone emulsions.

Compositions of the present invention are surprisingly effective in reducing the signs of photoaging and biological aging. Without wishing to be bound to a theory, compositions of the present invention are believed to obtain perceptible skin benefits (e.g. smoother, more evenly-toned skin with reduced appearance of fine lines and wrinkles) due to their multifunctional nature—providing moisturization and conditioning to the skin, decreasing trans-epidermal water loss as well as exfoliating and cleansing. The exfoliating properties of the biologically active extract(s) of the present invention increase turnover of epidermal cells.

Compositions of the present invention surprisingly and unexpectedly have been found to improve and maintain cell membrane integrity and connective tissue health. Without wishing to be bound to a theory, the high content of unsaturated C₁₈ fatty acids (relative to total fatty acids in the extract) are believed to act as building blocks for cell membranes. Additionally, the high relative ratio of 9-octadecanoic acid compared to other fatty acids in the composition of the present invention is believed to reduce damage from inflammatory processes.

Compositions of the present invention also comprise arabinose, rhamnose, fructose, glucose and galacturonic acid. Without wishing to be bound by a theory, these sugars and sugar derivatives are believed to be components in the synthesis of glycosaminoglycans and their aldosamine components (e.g., N-acetyl glucosamine). The glycosaminoglycans, in turn, are believed to help increase the synthesis of connective tissue, including collagen and elastin, and are thus useful in treating a variety of dermatologic conditions, including those caused by environmental or chronologic aging, as well as for maintaining connective tissue health. By maintaining connective tissue health is meant reducing the rate of degradation of connective tissue or otherwise limiting processes that have deleterious effects on connective tissue and its components.

Among the uses of the compositions of the present invention in the practice of dermatology, non-limiting examples include the following conditions: reduced skin elasticity; decreased skin firmness; loss of skin moisture; dry skin; pruritus; blotches; wrinkles; lentigines; age spots; melasmas; hyperpigmented skin; hyperkeratotic skin; skin atrophy; senile purpura; psoriasis; eczema; inflammatory dermatoses; spider veins; keratosis. Compositions of the present invention may be also used in the treatment of dry, thinning or brittle hair or nails. Additional dermatologic conditions which can be treated with compositions of the present invention include those described in Freedberg et al., Fitzpatrick's Dermatology in General Medicine (6^{th} Edition, 2003), Kerdel, et al., Dermatologic Therapeutics (2005), and Hardman et al., Goodman & Gilman's: The Pharmacological Basis of Therapeutics (10^{th} Edition, 2001)

In one embodiment, the compositions of the present invention contain at least one of an antioxidant, an essential fatty acid or a glycosaminoglycan. In a particularly preferred embodiment of the present invention, the at least one antioxidant, essential fatty acid or glycosaminoglycan is derived from, or contained in, the extract of D. zibethinus.

Preferred antioxidants are selected from the group consisting of: retinoids including retinol, retinal, retinol esters, retinyl propionate, retinoic acid, retinyl palmitate, and derivatives thereof; ascorbic acid, derivatives of ascorbic acid and mixtures thereof; tocopherol, derivatives of tocopherol and mixtures thereof; superoxide dismutase; polyphenols selected from the group consisting of phenolic acids, flavonoids, stilbenes and lignans; carotenoids selected from the group consisting of beta-carotene, alpha-carotene, lutein, zeaxanthin, lycopene, and cryptoxanthin; Coenzyme Q10, derivatives of Coenzyme Q10, and mixtures thereof; sulfhydryl compounds, including glutathione.

Preferred glycosaminoglycans are hyaluronic acid and chondroitin sulfate.

Preferred essential fatty acids include octadecenoic acids, octadecadienoic acids and octadecatrienoic acids.

The CTFA Dictionary describes a wide variety of non-limiting cosmetic and pharmaceutical ingredients that, optionally, are suitable for use in compositions of the present invention. Examples of these ingredient classes include: abrasives, exoliants, absorbents, astringents, antimicrobial agents, antioxidants, anti-inflammatory agents, vitamins, trace minerals, film formers and other polymeric materials that increase the substantivity of the compositions of the present invention to the skin, humectants, moisturizers, pH adjusters, skin-conditioning agents, skin soothing and/or healing agents, anti-acne agents, skin bleaching and lightening agents, external analgesics, sunscreen actives. Other examples of cosmetic and/or pharmaceutical ingredients which are suitable for use in compositions of the present invention are disclosed in U.S. Pat. No. 6,492,326 and U.S. Patent Publication No. 2005/0142095, the disclosures of which are incorporated herein by reference.

The following examples are further illustrative of the present invention. The components and specific ingredients are presented as being typical, and various modifications can be derived in view of the foregoing disclosure within the scope of the invention.

Two clinical studies are designed to observe the effect of D. zibethinus extract on patients with photodamaged and biologically aged skin. The first involves topical products, the second oral supplements. In the first study, fine lines and wrinkles are each measured using silicone replicas obtained from the outer canthus area of both sides of the face. Skin thickness is determined using ultrasound imaging of the cheek area. Ultrasound measures the relative density of the skin. An improvement in skin firmness is indicated by an increase in denser areas and decrease in the thinner areas, the former being associated with higher concentrations of collagen and elastin. Skin firmness is measured using a Dermalab (Cortex Technology, Denmark). In addition to the analytical measurements described above, clinical evaluations are performed by a trained technician and panelists complete a self-administered questionnaire regarding perceived improvements. In the second study, improved cell membrane integrity is measured using bioelectrical impedance; more particularly phase angle is evaluated using Bioelectrical Body Composition Analyzer from RJL Systems (Clinton Twp., Mich.).

In the first study, two water-in-oil skin creams, designated A and B, containing the following ingredients are administered:

| Ingredients | % Weight |
|---|---|
| Phase A | |
| Lauryl PEG/PPG-18/18 Methicone | 2.0 |
| Cyclomethicone and Dimethicone Crosspolymer | 5.0 |
| Mineral Oil | 3.0 |
| Extract of D. zibethinus | 5.0 |
| C12-15 Alkyl Benzoate | 5.0 |
| C30-45 Alkyl Methicone | 3.0 |
| Phase B | |
| Water | 63.6 |
| Sodium chloride | 1.0 |
| Preservative | 0.25 |
| Phase C | |
| Cyclomethicone | 6.0 |
| Trisiloxane and Dimethicone | 6.0 |
| Phase D | |
| Fragrance | 0.15 |

Formulas A and B differ only in that A contains crude, pressed extract, whereas B contains purified extract. Formula C serves as control. It is the same as Formulas A and B, except that it contains no extract; water is added in place of the extract. All three emulsions are prepared according to the following procedure. Heat phase A ingredients to 80° C. Mix ingredients of phase B together and heat to 80° C. Slowly add phase B to phase A and mix well. Homogenize using a high shear mixer. Allow the mixture cool to room temperature. Add ingredients of phase C one after another and mix with the same speed. Continue mixing for an additional 15 minutes.

Thirty test panelists, all female, ages 35-59 with Fitzpatrick Types I-III skin and minimal signs of photoaging, are recruited. The panelists are divided into three groups of ten and are provided with a skin cream to be applied twice daily for eight consecutive weeks. The panelists are also provided with the same facial cleansing product to be used for the duration of the study. Three sets of measurements are taken: baseline (prior to treatment); mid-course (after four weeks) and at the completion of the study. Expected results for fine lines and wrinkles, skin density (and thin skin) and skin firmness are as follows:

Relative to baseline, panelists using Sample A (crude extract) and Sample B (purified extract) show an average 15% and 20% reduction in fine lines and wrinkles at four weeks, respectively. At eight weeks, these panelists show further reductions—20% and 25%, respectively. Panelists receiving the control cream (Formula C) show only a 10% decrease, possibly due to improved skin hydration.

After four weeks, panelists using Sample A show a 15% increase in denser areas, and a 20% increase after 8 weeks. Sample B users show increased skin density of 25% and 30%, at four and eight weeks respectively. Controls, in contrast, show only a 10% increase in density. Thinner areas, likewise decrease with application of the extract. In panelists using Sample A, thin skin decreases at four and eight weeks respectively by 12% and 10%. Sample B users measure, on average, a decrease in thin skin of 15% and 12%. Controls show a decrease in skin thinness of 8% and 6%.

Skin firmness increases with Sample A by 10% and 20% at weeks four and eight; with Sample B, the increase is 15% and 25%. The control group shows an increase of 7%.

In the second study, a group of forty panelists, men ages 35-59 are recruited. A test group of twenty is administered an oral, twice-daily, 500 mg caplet comprising 50 mg of a purified extract of D. zibethinus, with the remainder of the caplet containing the following pharmaceutical excipients: microcrystalline cellulose, 400 mg; magnesium stearate, 25 mg; hydrated silica, 25 mg. A baseline phase angle measurement is taken and is compared with the following age/gendered normalized values:

| Phase Angle (degrees) | Assessment |
|---|---|
| 9.25-10.0 | Above Average |
| 7.75-9.25 | Average |
| Below 7.75 | Below average |

After eight weeks, phase angle is again measured. Expected results are as follows: Subjects who received the composition of the present invention have an increase in phase angle on average of about one degree, indicating improved cell membrane integrity.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth hereinabove but rather that the claims be construed as encompassing all the features of patentable novelty which reside in the present invention, including all features which would be treated as equivalents thereof by those skilled in the art to which the invention pertains.

1. A pharmaceutical or dermatocosmetic composition comprising a therapeutically-effective amount of a biologically-active extract of D. zibethinus, or active fraction thereof.

2. The composition of claim 1 where the biologically-active extract of D. zibethinus, or active fraction thereof, comprises (i) 9-octadecanoic acid in a ratio of at least about 2:3 to the total fatty acid content of the extract and (ii) a mixture of polysaccharides comprising at least one of arabinose, rhamnose and galacturonic acid.

3. The composition of claim 2 wherein 9-octadecanoic acid is present in a ratio of at least about 1:1 to the total fatty acid content of the extract.

4. A method for increasing phase angle in a person comprising administering to a person in need thereof the composition of claim 1.

5. A method of increasing phase angle in a person comprising administering to a person in need thereof the composition of claim 2.

6. The method of claim 4 where phase angle increases by at least 1% after administration of the composition of claim 1.

7. The method of claim 4 where phase angle increases by at least 5% after administration of the composition of claim 1.

8. The method of claim 4 where phase angle increases by at least 10% after administration of the composition of claim 1.

9. A method for treating or reducing the appearance of fine lines and wrinkles on the skin comprising administering to a person in need thereof the composition of claim 1.

10. A method for treating or reducing the appearance of fine lines and wrinkles on the skin comprising administering to a person in need thereof the composition of claim 2.

11. The method of claim 4 where the composition of claim 1 is administered orally.

12. The method of claim 5 where the composition of claim 2 is administered orally.

13. The method of claim 4 where the composition of claim 1 is administered topically.

14. The method of claim 5 where the composition of claim 2 is administered topically.

15. The method of claim 9 where the composition of claim 1 is administered orally.

16. The method of claim 10 where the composition of claim 2 is administered orally.

17. The method of claim 9 where the composition of claim 1 is administered topically.

18. The method of claim 10 where the composition of claim 2 is administered topically.

19. The composition of claim 1 further comprising at least one of an antioxidant, a glycosaminoglycan or an essential fatty acid.

20. The composition of claim 2 further comprising at least one of an antioxidant, a glycosaminoglycan or an essential fatty acid.

21. The composition of claim 19 wherein the at least one antioxidant, glycosaminoglycan or essential fatty acid is derived from, or contained in, the extract of D. zibethinus.

22. The composition of claim 20 wherein the at least one antioxidant, glycosaminoglycan or essential fatty acid is derived from, or contained in, the extract of D. zibethinus.
